(19) United States
(12) Patent Application Publication
(10) Pub. No.: US 2006/0129119 A1
(43) Pub. Date: Jun. 15, 2006

(54) Absorbent undergarment with disposal feature

(75) Inventor: Annastacia Jane Kistler, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/013,063

(22) Filed: Dec. 15, 2004

(51) Int. Cl. A61F 13/15

(52) U.S. Cl. 604/385.14; 604/395

(57) ABSTRACT

An absorbent article (2) includes a first body panel (4) having a first waistband edge (20) and laterally opposed first side portions (24); and a separately provided second body panel (6) having a second waistband edge (18) and laterally opposed second side portions (28). A separately provided crotch member (50) is operatively connected between the first body panel (4) and second body panel (6). The first body panel (4) is operatively connected to the crotch member (50) along a contoured attachment zone (54) which thereby provides a pocket section (56) of the first body panel (4). The pocket section (56) is operatively unattached to the crotch member (50), and has a free-edge region (58) that provides an operative access opening into the pocket section (56). The free-edge region (58) of the pocket section (56) includes an operative, pocket fastener mechanism (74) that is operatively connected to a bodyside surface of the pocket section (56) of the first body panel (4).

### FIELD OF THE INVENTION

The present invention relates generally to a disposable article. More particularly, the invention pertains to a disposable undergarment having a distinctive disposability feature.

### BACKGROUND

Disposable absorbent articles have been configured in many different forms, such as infant diapers, children's training pants, feminine care articles, adult incontinence articles, and disposable pants or panties. For example, disposable absorbent articles have been configured as a pant-type, pull-on garment, or as a diaper-type product that is drawn up between the legs and fastened about the waist with various fastening systems.

Particular arrangements of the absorbent articles have included a first body panel, a second body panel and a separately provided crotch member that interconnects between the body panels. Side portions of the body panels have been bonded or otherwise connected together to form a three-dimensional undergarment.

Various disposable systems and mechanisms have been incorporated into the disposable articles. For example, auxiliary adhesive tapes and other auxiliary fasteners have been employed to hold the article in a rolled or folded condition that is suitable for convenient disposal. Some articles have included integrally formed or separately provided bags or pouches that could be employed to hold a soiled article for disposal. Other articles have included extending panel members that could be manipulated to partially envelop rolled or folded portions of a soiled article for disposal.

Conventional disposal systems, such as those described above, have been cumbersome to use or have not been sufficiently effective. The auxiliary fasteners, for example, have not provided a sufficient level of discretion and have not adequately secured edge regions of the soiled articles. The bags or pouches have required an excessive handling of the soiled article. Similarly, the technique of partially enveloping rolled or folded sections of the article have required an excessive manipulation of the soiled article.

As a result, there has been a continuing need for more effective systems that can be employed to hold a soiled article in condition for disposal. In particular, there has been a continuing need for an improved system that can be more readily employed to hold a soiled undergarment in a desired disposal condition.

### BRIEF DESCRIPTION OF THE INVENTION

Generally stated, the present invention provides a disposable article that includes a distinctive system for efficiently holding the soiled article in a condition for convenient disposal. The article includes a first body panel having a first waistband edge and laterally opposed first side portions; and a separately provided second body panel having a second waistband edge and laterally opposed second side portions. A separately provided crotch member is operatively connected between the first body panel and second body panel. The first body panel is operatively connected to the crotch member along a contoured attachment zone which thereby provides a pocket section of the first body panel. The pocket section is operatively unattached to the crotch member, and has a free-edge region that provides an operative access opening into the pocket section. The free-edge region of the pocket section includes an operative, pocket fastener mechanism that is operatively connected to a bodyside surface of the pocket section of the first body panel. As a result, the present invention can provide an improved disposal system that can be more readily employed to hold a soiled article (e.g. undergarment) in a desired disposal condition. The disposal system can be distinctively configured to be integral to the disposable article, and can be readily employed to discreetly place and hold the soiled article in the desired disposal configuration.

The foregoing paragraph has been provided by way of general introduction, and is not intended to limit the scope of the following claims. The presently preferred configurations, together with further advantages, aspects and features, will be best understood by reference to the following detailed description taken in conjunction with the accompanying drawings.

### BRIEF DESCRIPTION OF THE DRAWINGS

FIG. 1 is a partially cut away, plan view of the bodyside of a representative, absorbent garment.

FIG. 2 is a schematic view of a representative, transverse cross-section taken along line 2-2 in the absorbent garment illustrated in FIG. 1.

FIG. 3 is a schematic view of a representative, transverse cross-section taken along line 3-3 in the absorbent garment illustrated in FIG. 1.

FIG. 4 is a schematic view of a representative, transverse cross-section taken along line 4-4 in the absorbent garment illustrated in FIG. 1.

FIG. 5 is a plan view of the bodyside of a representative, absorbent garment having a pocket section that can help hold the garment in a desired disposal configuration.

FIG. 5A is a plan view of the garment-side of the representative, absorbent garment having a pocket section that can help hold the garment in a desired disposal configuration.

FIG. 6 shows a representative article in which the crotch member has been turned back on itself along a crotch-fold, such that the side, ear portions of the first body panel are cooperatively adjacent to the side, ear portions of the second body panel.

FIG. 6A representatively shows the article of FIG. 6 wherein the aligned, adjacently positioned ear portions at one lateral side of the article have been turned back at a first ear-fold in a generally lateral movement in a direction which rotates away from the pocket section of the article.

FIG. 6B representatively shows the article of FIG. 6A wherein the aligned, adjacently positioned ear portions at the second, laterally-opposed side of the article have been turned back along a second ear-fold 92a in a lateral movement in a direction which rotates away from the pocket section.

FIG. 6C representatively shows the article of FIG. 6B wherein the previously turned back portions of the body panels have been further turned back in a generally longitudinal movement which pivots in a direction away from the pocket section, and the distal portions of the body panels have been arranged to extend beyond the location of the crotch-fold.

FIG. 6D representatively shows the article of FIG. 6C wherein the distal portions of the body panels have been inserted into the pocket section past the pocket free-edge region, and operatively secured with the pocket fastener.

### DETAILED DESCRIPTION OF THE INVENTION

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

It should also be understood that the term “longitudinal,” as used herein, means of or relating to length or the lengthwise direction, and in particular, the direction running between the front and back of the wearer. The term “laterally,” as used herein means situated on, directed toward or running from side to side, and in particular, a direction running to the left and right of a wearer. The terms “upper,” “lower,” “inner”, and “outer” as used herein are intended to indicate the direction relative to the wearer wearing an absorbent garment over the crotch region, while the terms “inboard” and “outboard” refer to the directions relative to a longitudinally-extending centerline 8 of the garment and the absorbent composite 50. For example, the terms “inner” and “upper” refer to a “bodyside,” which means the side relatively closer to or facing towards the body of the wearer; while the terms “outer” and “lower” refer to a “garment-side” facing towards a wearer's outer clothing.

The term “bodyside” should not be interpreted to mean in contact with the body of the wearer, but rather simply means the side that would face toward the body of the wearer, regardless of whether an undergarment is actually being worn and regardless of whether there are or may be intervening layers between the component and the body of the wearer. Likewise, the term “garment-side” should not be interpreted to mean in contact with the garments of the wearer, but rather simply means the side that faces away from the body of the wearer, and therefore toward any outer garments that might also be worn, regardless of whether the undergarment is actually being worn by a wearer, regardless of whether any such outer garments are actually worn and regardless of whether there may be intervening layers between the component and any outer garment.

The phrases “removably attached,” “removably attaching,” “removably connected,” “removably engaged,” “releasably attached,” “releasably connected,” or “releasably engaged,” and variations thereof, refers to two or more elements being connected or connectable such that the elements tend to remain connected absent a separation force applied to one, both or all of the elements, and where the elements are capable of being separated upon the application of a separation force. The required separation force is typically beyond that encountered while wearing the article.

The phrases “fixedly secured,” “fixedly engaged,” “fixedly attached,” “fixedly connected,” and variations thereof, refers to two or more elements being connected or connectable such that they are not disconnected or otherwise separated, and are not intended to be separated or disconnected, during the normal operation and use of the article.

The term “web” refers to a continuous stream of material, whether made from one or more layers or substrates, and regardless of whether it may have non-continuous, discrete items disposed thereon.

The terms “connecting,” “coupled,” “attached,” and “secured,” and variations thereof, broadly covers two or more items connected directly one to the other, or by way of one or more intervening members or components.

The term “coform” as used herein is intended to describe a blend of meltblown fibers and cellulose fibers that is formed by air forming a meltblown polymer material while simultaneously blowing air-suspended cellulose fibers into the stream of meltblown fibers. The meltblown fibers containing wood fibers are collected on a forming surface, such as provided by a foraminous belt. The forming surface may include a gas-pervious material, such as spunbonded fabric material, that has been placed onto the forming surface.

Disposable absorbent articles, such as disposable absorbent undergarments, have included a first body panel, a second body panel and a separately provided crotch member that interconnects between the body panels. Side portions of the body panels have been bonded or otherwise connected together to form a three-dimensional undergarment. Examples of absorbent undergarments are described in detail in U.S. Patent Application Publication No. 20040060648 entitled THREE-PIECE DISPOSABLE UNDERGARMENT AND METHOD FOR THE MANUFACTURE THEREOF by R. Thorsen et al., which was published Apr. 1, 2004 (attorney docket No. 17,798); and U.S. Patent Application Publication No. 20040122412 entitled ABSORBENT GARMENT HAVING A BODY CONFORMING ABSORBENT COMPOSITE by M. Morman et al., which was published Jun. 24, 2004 (attorney docket No. 18,019). The entire disclosures of these documents are incorporated herein by reference in a manner that is consistent herewith.

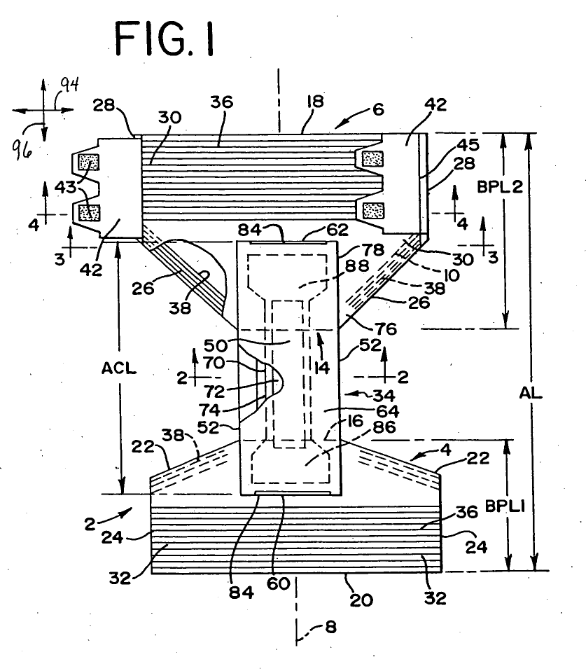

With reference to FIGS. 1 through 5A, an absorbent article 2 has a laterally-extending, transverse cross-direction 94, and a lengthwise-extending, longitudinal-direction 96. The article includes a first body panel 4, which has a first waistband edge 20 and laterally opposed first side portions 24; and a separately provided second body panel 6, which has a second waistband edge 18 and laterally opposed second side portions 28. A separately provided crotch member 50 is operatively connected between the first body panel 4 and second body panel 6. At least one of the first and second body panels 4,6 is operatively connected to the crotch member 50 along a contoured attachment zone 54 which thereby provides a pocket section 56 of each of the desired body panels. In the representatively shown arrangement, the pocket section 56 is formed with the first body panel 4. The pocket section 56 is operatively unattached to the crotch member 50, and has a free-edge region 58 that provides an operative access opening into the pocket section 56. The free-edge region 58 of the pocket section 56 includes an operative, pocket fastener mechanism 74 that is operatively connected to a bodyside surface of the pocket section 56 of the first body panel 4 and/or second body panel 6. Optionally, the pocket fastener mechanism may be operatively connected to an extending tab member, which is in turn operatively connected to a bodyside or garment-side surface of the pocket section 56.

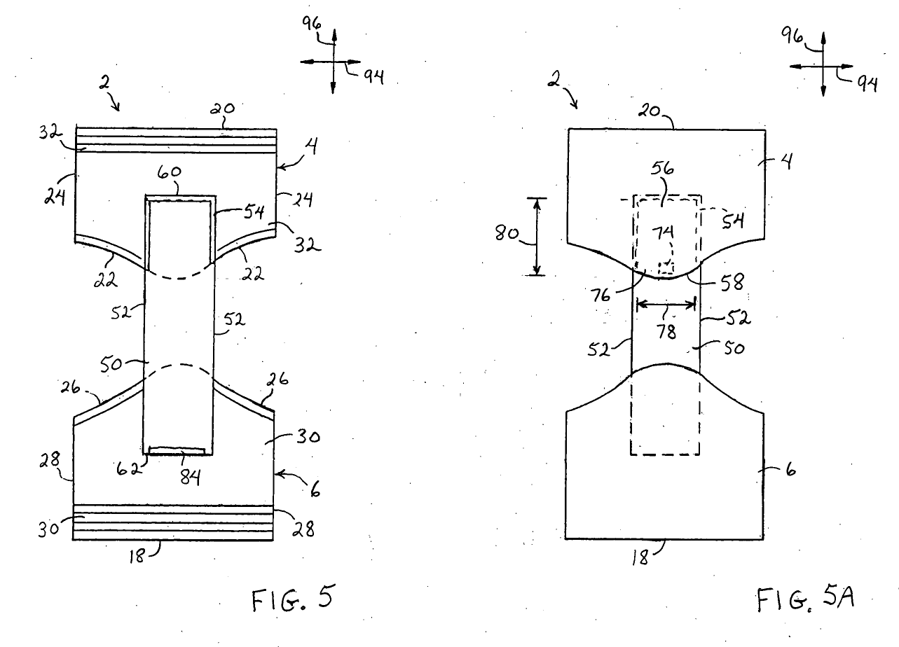

In the present disclosure, the term “body panel” refers to the portion(s) of the absorbent garment, whether made of one or more layers or substrates or of one or more pieces or components that is/are fitted circumferentially around at least the waist region of the wearer, including for example the wearer's lower back, buttock, hips and abdomen. The first body panel 4 can provide a front or rear body panel, as desired. Similarly, the second body panel 6 can appropriately provide a cooperative rear or front body panel. The first and second body panels 4,6 each have an inner, bodyside surface 10; an outer, garment-side surface 12; and a panel length that is less the overall length of the absorbent garment. Each of the first and second body panels has outer side edges, and has first and second longitudinally opposed terminal end edges which form crotch edges 16, 14, and waist edges 20, 18 respectively. As representatively shown in FIGS. 1 and 5, the side edges can include a tapered edge 22, 26 and an outboard edge 24, 28 formed along the outer periphery of laterally opposed ear portions 30, 32. Alternatively, the front and rear body panels may not include a tapered side edge, but may include a terminal crotch edge that is substantially rectilinear.

The first terminal edges 14, 16 of the first and second body panels are longitudinally spaced apart to form a gap 34 therebetween in the crotch region of the garment, while the second terminal edges 20, 18 of the first and second body panels form front and back waist edges respectively. As shown, a plurality, meaning two or more, of laterally extending elastic elements 36 can be secured to each of the first and second body panels. Likewise, one or more leg elastic elements 38 can be secured along the tapered side edge of the body panels to form a gasket with the leg of the wearer.

For example, each panel can include an elasticized composite panel material comprising two non-woven substrates 40 with at least one elastic element 36 or a plurality of elastic elements 38 sandwiched therebetween. As representatively shown, the elastic elements can be elastic strands, and the strands can be positioned in the waist regions and along the leg perimeters. A portion of the leg elastic elements 38 can extend under a side margin of the crotch member 50. The placement of the panel leg elastic elements further inward along the side edge can help provide for improved fit and performance of the garment. In a particular configuration, a plurality of elastic elements can be spaced across substantially the entire waist portion of the front and rear body panel 4, 6, although they may be spaced across a lesser length. In an optional arrangement, an elastomeric layer, such as a film, can be disposed between the substrates to provide one or more selected elastomeric elements to form the desired, elasticized composite panel. In a particular aspect, the body panel edges 22 and 26 can cooperate with a portion of an associated side edge 52 of the crotch member 50 to operatively form a corresponding leg opening region of the article. The elastic elements 36, 38 can extend along the waist portion of the body panels and along the tapered terminal edges of the body panels to help form a laterally-opposed pair of leg opening regions of the article.

In a particular aspect, the front body panel can have a “non-elasticized” area wherein there are no elastic elements, or other elastic or elastomeric backing members, incorporated therein or making up any portion of the thickness or cross-section of the body panel at that area. It should be understood, that in an alternative arrangement, one or more separate waist band members, with or without elastic elements, can be secured to one or both of the first and second body panels, preferably along the upper terminal edges 20, 18 thereof. Likewise, one or more separate leg band members can be secured to one or both of the rear and front body panels along the leg open portions 22, 26 adjacent the leg opening regions. Alternatively, one or both of the body panels can be formed without any elastic elements.

The various waist and leg elastic elements can be formed from rubber or other elastomeric materials. One suitable material is a LYCRA elastic material. For example, the various elastic elements can be formed of LYCRA XA Spandex 540, 740 or 940 decitex T-127 or T-128 elastics available from E.I. DuPont De Nemours and Company, having an office in Wilmington, Del.

In a particular aspect, each body panel 4, 6 can be formed as a composite, or laminate material, otherwise referred to as substrates or laminates, with the plurality of elastic strands sandwiched therebetween. For example, two or more layers may be bonded with various adhesives, such as hot melt, or by other techniques, including for example and without limitation, ultrasonic bonding and heat pressure sealing. In one embodiment, the two layers are made of a non-woven material such as a spunbond material, a bonded carded material or other known materials. It should be understood that the body panels can be made of a single layer or substrate of non-woven material, or can be comprised of more than two layers or substrates. Of course, it should be understood that other knitted or woven fabrics, non-woven fabrics, elastomeric materials, polymer films, laminates and the like can be used to form one or more of the body panel layers. The term “non-woven” web or material, as used herein, means a web having a structure of individual fibers or filaments that are interlaid, but not in an identifiable manner and without the aid of textile weaving or knitting, as in a knitted or woven fabric.

In another aspect, the body panel material can be secured to the elastic elements, such as strands or ribbons that have been elongated and retracted, such that the material is gathered when the elastic elements are relaxed. Alternatively, the material can be gathered and laminated to non-elongated elastic elements. In one embodiment, the body panel includes a gathered elastic laminate made from nonwoven base sheets bonded with elongated elastic elements sandwiched therebetween.

In various configurations, the body panel material may be substantially permeable to air or substantially impermeable to air. The body panel material also may be substantially liquid-permeable or substantially liquid-impermeable. In particular arrangements, the body panel material may be substantially nonelastomeric. In other aspects, the body panels can include an elastomeric material that is elastomerically stretchable at least along the lateral article width. Examples of such elastomeric composite materials can include a vertical filament laminate (VFL), neck-bonded-laminate (NBL), a stretch-bonded-laminate (SBL), a necked-stretch bonded laminate (NSBL) or a necked-thermal laminate, or the like, as well as combinations thereof. Exemplary NBL, SBL, and NSBL materials are described in U.S. Pat. Nos. 5,226,992; 4,981,747; 4,965,122; 5,336,545; 5,385,775; 4,720,415; 4,789,699; 4,781,966; 4,657,802; 4,652,487; 4,655,760; 5,116,662 and 5,114,781, all of which are hereby incorporated herein by reference. Exemplary VFL materials are described in U.S. Provisional Patent Application Ser. No. 60/204,307, filed May 15, 2000 and entitled “Method and Apparatus for Producing Laminated Articles,” and PCT application WO 01/88245 A2 (attorney docket No. 14590), both assigned to Kimberly-Clark Worldwide, Inc., the assignee of the present application, with the entire disclosures of both being hereby incorporated herein by reference. Such laminates can provide an improved combination of cloth-like feel and elastomeric stretchability. The body panels can be composed of materials that are elastic or elastomeric and exhibit biaxial stretch characteristics or MD/CD stretch characteristics, or that are extensible composites. Additional waist and leg elastic elements can be added to, but are not necessarily required by, the body panels.

In particular configurations, the entirety of the body panels can be elasticized, such that the entire body panel conforms to the body of the wearer without any spacing between the body panel and the wearer's body. As a result, the body panels can help avoid the bulkiness that can arise during the wearing of a non-elasticized material.

In particular aspects, the body panels can be breathable, cloth-like, multi-directional nonwoven laminates with stretch and/or extensible properties. In other aspects, the non-woven layers can be pre-necked, preferably between about 10% and about 80%, in the longitudinal direction, which provides extensibility in the longitudinal direction with minimum force.

The terms “extensible,” “extensibility,” and variations thereof as used herein means capable of being extended, and providing a selected elongation, for example between about 5% and about 70%, when subjected to an applied tensile force. The body panel also is preferably capable of providing a selected, sustained deformation when subjected to an applied tensile force and then allowed to relax for a selected time period beginning immediately after removal of the tensile force. Preferably, the sustained deformation is a substantially permanent deformation. The selected elongation and sustained deformation preferably occur at least along the longitudinal direction of the garment, although it should be understood that it also could occur along the lateral direction, or both. Various extensible materials, and other acceptable materials that can be used for the body panels are described for example in U.S. Pat. No. 6,217,563, issued Apr. 17, 2001 to Kimberly-Clark Worldwide, Inc., the same Assignee as the present application, the entire disclosure of which is hereby incorporated herein by reference in a manner that is consistent herewith.

The extensibility of the non-woven material can provide an increase in its surface area without incurring the retractive force generated by elastomeric materials. In particular aspects, the body panel can be extensible in at least the cross-direction, or longitudinal direction, with the material providing an elongation of at least about 1 cm when subjected to a tensile stress of 11.8 grams per cm. In addition, the body panel can preferably provide a substantially permanent deformation of at least about 20% when it is subjected to a tensile stress of 19.70 grams per cm and is then allowed to relax under a zero applied stress for a period of 1 minute. Of course, it should be understood that the body panel can also be made extensible in both the lateral direction and longitudinal direction.

In a particular configuration, the front and rear body panels 4, 6 can include non-woven laminates of two layers of longitudinally extensible 0.60 osy polypropylene spunbond material with elongated strands of LYCRA elastic sandwiched between the spunbond layers and thereafter adhesively bonded. In particular, the body panel material is necked in the cross-direction. As used herein, the term “necked,” and variations thereof, refers to any material that has been constricted in at least one dimension by applying a tensioning force in a direction that is perpendicular to the desired direction of neck-down. Processes that may be used to constrict a material in such a manner include, for example and without limitation, drawing or stretching processes. Elastics are then elongated in the machine-direction and secured to the body panel material. The elastics are then allowed to retract so as to gather the necked spunbond material along the machine-direction, thereby creating an elastically gathered, non-woven body panel with operative extensibility. The term “gather,” and variations thereof, as used herein means puckered, or contracted into folds or wrinkles, which should be understood as including micro-pleats. In this way, the body panel can be elongated in both the longitudinal and lateral direction to conform to the body of the wearer when the garment is worn. In particular, as the wearer pulls the garment up over the hips, the non-woven laminate body panels can stretch in the lateral direction while the leg regions of the front and rear body panels conform to the crotch and body lines of the wearer. At the same time, the body panel material can extend in the longitudinal direction to conform to the buttocks and stomach of the wearer. The extensibility of the body panels helps the body panels follow the natural curvature of wearer's body to provide conformance thereto. In a desired configuration, the spacing between the laterally extending elastic elements 36 incorporated will increase as the body panels extend in the longitudinal direction.

In a particular aspect, the non-woven material of a body panel 4, 6 can be substantially hydrophobic, and may optionally be treated with a surfactant or otherwise processed to impart a desired level of wettability and hydrophilicity. In one particular embodiment of the invention, the body panel is a nonwoven, wire-weave spunbond polypropylene fabric composed of about 1.6 denier fibers formed into a web having a basis weight of about 0.6 osy (about 20.3 g/m²). One suitable non-woven material is the Corinth 0.6 osy (about 20.3 g/m²), 1.6 dpf wireweave, nonwettable Metallocene (EXXON ACHIEVE 2854 PP) spunbond material manufactured by Kimberly-Clark Corporation, the assignee of the present application.

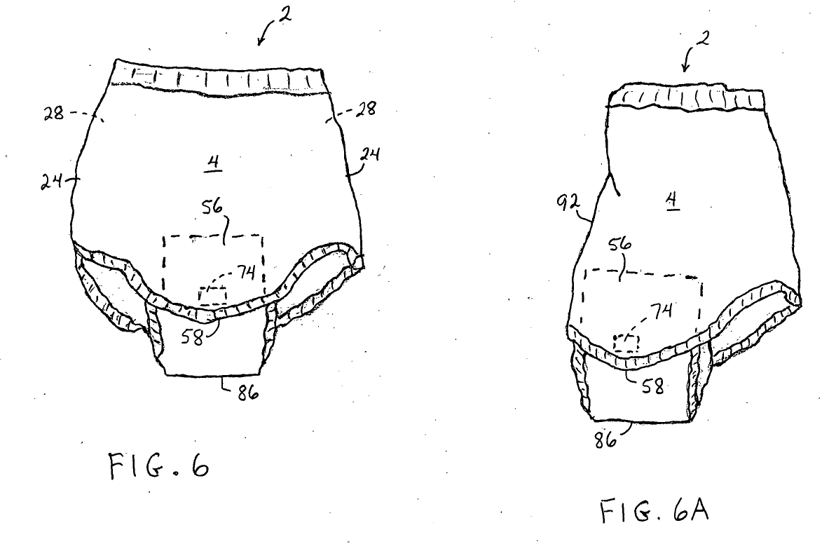

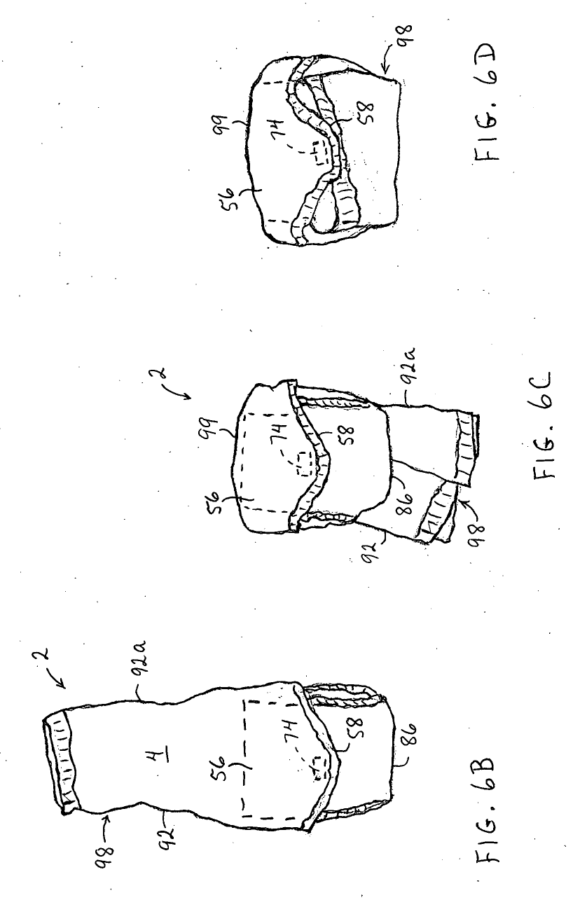

Referring to FIG. 6, the crotch member 50 that connects between the front and rear body panels 4, 6 can be folded or otherwise turned back on itself, such that the first side portions 24 of the first body panel 4 can be operatively connected to the second side portions 28 of the second body panel 6 to provide an article waistband that is configured to substantially encircle a wearer's waist. As representatively shown, the side edge portions 24, 28 of the first and second body panels 4, 6 can be operatively aligned to a position wherein the cooperating side edge portions can be fixedly secured at a seam. The seam can be formed by bonding, sewing or otherwise attaching the side edges.

Alternatively, the product can have an “open” condition, wherein the body panels can be releasably secured with one or more fastening members 42. For example, the fastening members can be attached to the second (e.g. back) body panel 6 at an attachment location that is spaced inboard from the side edge of the body panel. Additionally, the fastening members 42 can extend relative to the outboard side edge of the second body panel. An operative landing member can be formed on or secured to the first (e.g. front) body panel 4 to receive a refastenable portion 43 of the fastening member. For example, a nonwoven material or other “loop” material may be operatively constructed and located on the first body panel.

Alternatively, the body panels 4,6 can be cooperatively attached along their side edge portions 24, 28 to produce a pair of laterally opposed side seams and provide an article having a generally, three-dimensional form. Optionally, the garment can include a combination wherein cooperating side edge portions are secured together to form a seam, and fastening members are configured to adjust the fit of the absorbent garment.

It should be understood that, in particular arrangements, the fastening members 42 may alternatively be secured to the first (e.g. front) body panel 4 at an operative attachment location 45 and configured to engage the second (e.g. back) body panel 6. Additionally, the fastener members may be configured to engage an appointed body panel along at least a portion of the appointed body panel that is not elasticized. In a desired arrangement, the fastening members can be fixedly secured to the outer, garment-side surface of the front and/or rear body panels, and configured to releasably engage the outer, garment-side surface of the front and/or rear body panels, although it should be understood that the fastening members may optionally be fixedly secured to an inner body-side surface of front and/or rear body panels and configured to releasably engage an inner, body-side surface of the front and/or rear body panels. Accordingly, the article can include at least one side fastener which is operatively connected to the side portion of at least one body panel, and is configured to secure the absorbent article about the waist of a wearer.

One or more lines of weakness can be provided along the front or rear body panel such that one or both of the body panels are breakable. The lines of weakness can comprise a perforation or other series of cuts, a thinning, breakage or separation of material, or a strip of a different kind of material bridging portions of the body panel that is more easily torn or broken than the other material thereof, which allow a wearer or the manufacturer to separate portions of the body panel. For example, the absorbent garment can be broken along the lines of weakness after the garment is applied to a wearer, or beforehand. In one embodiment, the fastening members are secured to the garment-side surface of the body panel.

When incorporated into an absorbent garment, the fastening members 42 may include at least one refastenable portion 43, such as an array of hook members, adhesives, such as pressure sensitive adhesives, buttons, zippers, snaps and other releasable and reattachable fastening devices. In various preferred arrangements, the fastening member includes one, two or more than two tab members. In one embodiment, the fastening members comprise a carrier member, which is fixedly secured to the side portions of the front body panel with adhesive bonds, sonic bonds, thermal bonds, pinning, stitching or other known types of attachment. In alternative embodiments, the fastening members can be fixedly secured to the rear body panel or to one or both of the front and rear body panels, for example, at the seam, as explained above.

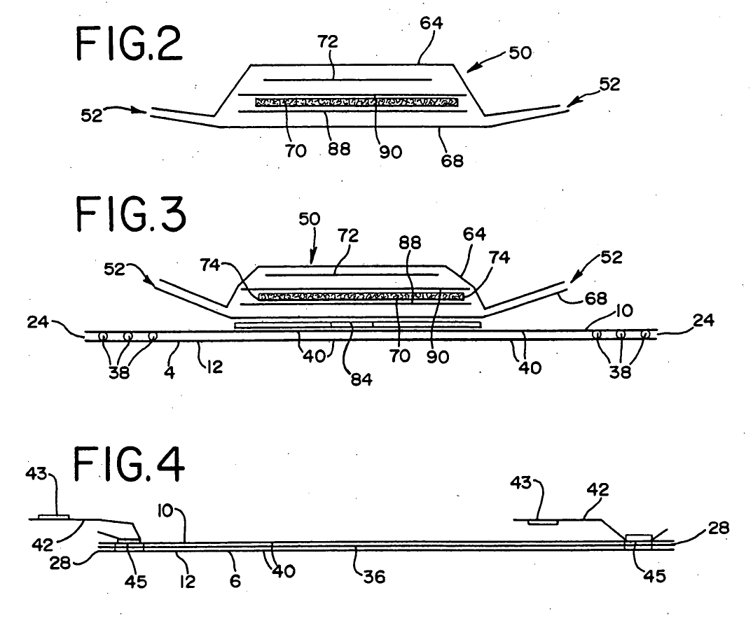

Referring to FIGS. 1 and 2, the crotch member 50 can be configured to include an absorbent composite or other absorbent body, and can have first and second opposed terminal end edges 60, 62. The crotch member and the provided absorbent composite can include any material or assembly capable of absorbing liquids or bodily exudates, and may be formed from a single material or component, for example a retention portion, or a combination of materials and/or components.

As representatively shown in FIGS. 1 through 4, the crotch member 50 can include an operatively, liquid permeable liner or topsheet 64, and an outer cover or backsheet 68. Desired configurations of the backsheet can be substantially liquid impermeable. An absorbent body, such as provided by a retention element 70, can be sandwiched or otherwise operatively disposed between the topsheet and the backsheet. It should be understood that the retention element can include any material or assembly capable of absorbing liquids or bodily exudates, and may be a single material or component, or can be formed as a composite of several components.

The topsheet, backsheet and other components of the crotch member 50 can be operatively connected with, for example, adhesive bonds, sonic bonds, thermal bonds, pinning, stitching or any other attachment techniques known in the art, as well as combinations thereof. For example, a uniform continuous layer of adhesive, a patterned layer of adhesive, a sprayed pattern of adhesive or any array of lines, swirls or spots of construction bonds may be used to join or otherwise connect together the topsheet and backsheet, or any of the other components described herein.

Additional layers, including for example, a transfer layer 72, can also be incorporated into the crotch member 50. In one embodiment, the transfer layer does not run the entire length of the crotch member and is shorter than the retention element. The topsheet 64 can be indirectly joined to the backsheet 68 by affixing the topsheet to intermediate layers, such as the transfer layer 72 or retention portion 70, which in turn is affixed to the backsheet. The crotch member also may include barrier cuffs, or leakage control shields, formed along the opposite longitudinally extending edges of the crotch member.

In various constructions, the topsheet 64 may include any material that can be configured to provide the topsheet with an operative level of liquid-permeability. The topsheet may be constructed with one or more layers of suitable materials, and may be a composite material. For example, the topsheet layer can include a woven fabric, a nonwoven fabric, a polymer film, a film-fabric laminate or the like, as well as combinations thereof. Examples of a nonwoven fabric include, spunbond fabric, meltblown fabric, coform fabric, a carded web, a bonded-carded-web, a bicomponent spunbond fabric or the like as well as combinations thereof. For example, the topsheet layer can include a woven fabric, a nonwoven fabric, a polymeric film that has been configured to be operatively liquid-permeable, or the like, as well as combinations thereof. Other examples of suitable materials for constructing the topsheet layer can include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

The topsheet can, for example, include a substantially hydrophobic material, and the hydrophobic material may optionally be treated with a surfactant or otherwise processed to import a desired level of wettability and hydrophilicity. In a particular arrangement, the topsheet can be a nonwoven, spunbond polypropylene fabric composed of about 2.8-3.2 denier fibers formed into a web having a basis weight of about 22 gsm and density of about 0.06 gm/cc. The fabric can be surface treated with an operative amount of surfactant, such as about 0.28% TRITON X-102 surfactant. The surfactant can be applied by any conventional means, such as spraying, printing, brush coating or the like, as well as combinations thereof.

The backsheet 68 may include a layer constructed of any operative material, and can be configured to be operatively liquid impermeable or liquid permeable, as desired. The backsheet may, for example, include a polymeric film, a woven fabric, a nonwoven fabric or the like, as well as combinations or composites thereof. For example, the backsheet may include a polymer film laminated to a woven or nonwoven fabric. In a particular feature, the polymer film can be composed of polyethylene, polypropylene, polyester or the like, as well as combinations thereof. Additionally, the polymer film may be micro-embossed. Desirably, the backsheet 68 can operatively permit a sufficient passage of air and moisture vapor out of the article, particularly out of the retention portion 70 while blocking the passage of bodily liquids. Additionally, the backsheet can desirably be a flexible material. The flexible material is compliant and will readily conform to the general shape and contour of the body of the wearer.

The backsheet can operatively prevent various bodily fluids and exudates from wetting or otherwise contaminating bedding items or items of outer garments that are typically worn by the wearer over the absorbent garment. In a particular aspect, the backsheet can include a film, such as a polyethylene film, having a thickness of from about 0.012 mm to about 0.051 mm. In other aspects, the backsheet can comprise a woven or nonwoven fibrous web layer, which is treated or constructed, partially or wholly, to impart the desired levels of liquid impermeability to selected regions that are adjacent to or proximate the absorbent retention portion. For example, the backsheet may include a gas-permeable, nonwoven fabric layer laminated to a polymer film layer which may or may not be gas-permeable. Other examples of fibrous, cloth-like backsheet materials can comprise a stretch thinned or stretch thermal laminate material composed of a 0.6 mil (0.015 mm) thick polypropylene cast film and a 0.7 ounce per square yard (23.8 gsm) polypropylene spunbond material (2 denier fibers). A material of this type has been employed to form the outercover of a HUGGIES ULTRATRIM Disposable Diaper, which has been commercially available from Kimberly-Clark Corporation. The backsheet can provide the outercover of the article, particularly in the crotch region. Optionally, however, the article may include a separate outercover component member, as disclosed herein, which is additional to the backsheet. The outercover can be joined, for example, to one or more of the absorbent composite and/or body panels as explained above.

The backsheet may include a micro-porous, “breathable” material which permits gases, such as water vapor, to escape from the absorbent garment while substantially preventing liquid exudates from passing through the backsheet. For example, the breathable backsheet may be composed of a microporous polynier film or a nonwoven fabric which has been coated or otherwise modified to impart a desired level of liquid impermeability. For example, a suitable microporous film can be a PMP-1 material, which is available from Mitsui Toatsu Chemicals, Inc., a company having offices in Tokyo, Japan; or an XKO-8044 polyolefin film available from 3M Company of Minneapolis, Minn. The backsheet may also be embossed or otherwise provided with a pattern or mafte finish to exhibit a more aesthetically pleasing appearance.

In various configurations of the invention, where a component, such as the backsheet is configured to be permeable to gas while having a resistance and limited permeability to aqueous liquid, the liquid resistant component can have a construction which is capable of supporting a selected hydrohead of water substantially without leakage therethrough. A suitable technique for determining the resistance of a material to liquid penetration is Federal Test Method Standard FTMS 191 Method 5514, 1978, or an equivalent thereof.

In one preferred embodiment, the backsheet is sufficiently impermeable to liquid and semi-liquid materials to substantially prevent the undesired leakage of waste materials, defined as exudates, including for example urine and feces. For example, the backsheet member can desirably support a hydrohead of at least about 45 centimeters (cm) substantially without leakage. The backsheet member can alternatively support a hydrohead of at least about 55 cm, and optionally, can support a hydrohead of at least about 60 cm, or more, to provide improved benefits.

The backsheet and/or outercover also can be extensible. In one preferred embodiment, the backsheet and/or outercover is capable of providing an elongation of at least about 1 cm when subjected to a tensile force of 11.8 g/cm, and further provides a substantially permanent deformation of at least about 20% when subjected to a tensile force of 19.70 g/cm and is then allowed to relax under a zero applied stress for a period of 1 minute.

For example, the extensible member can include a necked fiber, a creped fiber, a micro-pleated fiber, polymer films or the like, as well as combinations thereof. The fabrics may be woven or nonwoven materials, such as spunbond fabrics. One example of a suitable extensible material is a 60% necked, polypropylene spunbond having a basis weight of about 1.2 osy (about 41 g/m²).

The backsheet and/or outercover also can be expandable, for example when it has one or more folds, e.g., one or more z-folds (not shown), or can be both extensible and expandable. The term expandable as used herein means to enlarge or to increase the extent or area, lateral and/or longitudinal directions, e.g., by unfolding one or more folds.

The retention element 70 comprises an absorbent material, which can be any material that tends to swell or expand as it absorbs exudates, including various liquids and/or fluids excreted or exuded by the wearer. For example, the absorbent material can be made of airformed, airlaid and/or wetlaid composites of fibers and high absorbency materials. The fibers can, for example, include wood pulp fluff materials, such as Alliance CR-1654, or any combination of cross-linked fibers, hardwood, softwood, and synthetic fibers. The high absorbency materials have typically been referred to as superabsorbents, and the superabsorbents can be made from polyacrylic acids. For example, a suitable superabsorbent material is FAVOR 880 available from Stockhausen, Inc. of Greensboro, N.C., U.S.A. Airlaid and wetlaid structures may include binding agents, which are used to stabilize the structure. In addition, various foams, absorbent films, and superabsorbent fabrics can be used as an absorbent material. Various acceptable absorbent materials are disclosed in U.S. Pat. No. 5,147,343 for ABSORBENT PRODUCTS CONTAINING HYDROGELS WITH ABILITY TO SWELL AGAINST PRESSURE, U.S. Pat. No. 5,601,542 for ABSORBENT COMPOSITE, and U.S. Pat. No. 5,651,862 for WET FORMED ABSORBENT COMPOSITE, all of which are hereby incorporated herein by reference. Furthermore, the proportion of high-absorbency particles can range from about 0 to about 100%, and the proportion of fibrous material from about 0 to about 100%. Additionally, high absorbency fibers can be used such as Oasis type 121 and type 122 superabsorbent fibers available from Technical Absorbent Ltd., Grimsby, Lincolnshire, United Kingdom.

In various arrangements, the retention element 70 can include a single or dual layer of absorbent material. Alternatively, the retention portion can include a folded or multi-layered configuration. The retention element 70 preferably has a length substantially equal to, or slightly shorter than, the length of the crotch member 50. As shown in FIGS. 2 and 3, the retention portion can include one or more barrier layers 88, 90 attached to the absorbent material. In one arrangement, an upper barrier layer 90 can include a tissue substrate disposed adjacent the retention element. In addition, a lower barrier layer 88 can be provided by a tissue substrate disposed adjacent an opposite side of the retention element. Optionally, a tissue substrate can completely envelop the retention element. The retention element, and the overall absorbent composite, can have any operative shape. For example, the retention element can include a trapezoidal shape with first and second parallel terminal edges and non-parallel, linear side edges, wherein said first terminal edge is shorter than said second terminal edge.

As representatively shown in FIGS. 1 and 5-9, the first terminal end edge 60 of the crotch member 50 can overlie the first body panel 4 and the second terminal end edge 62 of the crotch member 50 can overlie the second body panel 6. Referring to the embodiment shown in FIG. 1, the opposite garment-side of the end regions of the crotch member 50, and in particular, the outer, garment-side surface of the crotch member, can be operatively secured to the bodyside surface of the first and second body panels 4, 6. Alternatively, the bodyside surface of the crotch member is secured to the garment-side surface of the body panels. It should be understood that the crotch member 50 can be secured to the body panels using any of the attachment mechanisms described above, including for example various adhesives, stitching or other bonding techniques.

The entire portion of the crotch member 50 overlapping the body panels 4, 6 can be attached to the body panels, or the crotch member can be attached to the body panels, for example by one or more selected attachment locations 84 at the terminal edges 60, 62 of the crotch member so as to allow the body panels to stretch from side to side and extend from front to back, or from the crotch to the waist.

As representatively shown in FIG. 1, the opposite garment-side of the crotch member 50, and in particular, the outer, garment-side surface of the backsheet 68, can be secured to the bodyside surface 10 of the longitudinally spaced first and second body panels 4, 6. As representatively shown in FIG. 1, only the terminal edges 60, 62 of the absorbent composite or other crotch member 50 can be connected to the bodyside surface of the body panels 4, 6.

Optionally, the body side surface of the top sheet of the absorbent composite can be secured to the garment-side surface of the body panels. The attachment location 84 can extend laterally across the entire width of the absorbent composite at the terminal edge thereof, or across only a portion thereof, as shown for example in FIGS. 1 and 3. The term “location” means any region, dot or position, and is not limited to lines, whether continuous or intermittent.

It should be understood that the crotch member 50 can be secured using any of the methods of attachment described above. Moreover, it should be understood that the absorbent composite or other crotch member 50 can be secured to the body panels with a continuous attachment along the selected terminal edges of the crotch member, or with a discontinuous attachment pattern, for example, such as provided by a plurality of laterally spaced apart attachment spots, or lines.

In the arrangement representatively shown in FIG. 1, the terminal edge regions 60, 62 of the absorbent composite are secured to the body panels 4, 6 intermediate the terminal crotch and waist edges 16, 20, 14, 18 of the body panels. As representatively shown, the terminal edges 60, 62 of the crotch member are longitudinally spaced from the terminal crotch edges 16, 14 of the front and rear body panels 4, 6 respectively, such that the crotch member has first and second unattached regions 86, 88 overlying the front and rear body panels respectively, with the “unattached regions” being defined as the portions of the absorbent composite that overlie the front and rear body panels but are not attached thereto.

With reference to FIGS. 5 and 5A, a particular aspect of the invention can have a configuration in which the first body panel 4 is operatively connected to the crotch member 50 along the contoured attachment zone 54 to thereby provide an operatively unattached pocket section 56 of the first body panel. The contoured attachment zone 54 can have any operative shape or form. The contour can be linear, curved, angular, rectilinear, curvilinear or the like, as well as combinations thereof. The contoured attachment zone 54 may or may not substantially follow the shape of the corresponding, longitudinal end-region of the crotch member 50. As representatively shown, for example, the contour of the attachment zone 54 can be generally U-shape and can have a generally angular form. Additionally, the contoured attachment zone 54 can extend along the terminal end edge 62 of the crotch member, and along the portions of the crotch member, side edges 52 that overlie their corresponding body panel (e.g. body panel 6).

The pocket section 56 is operatively unattached to the crotch member 50 and has a free edge region 58 that provides an operative access opening into the pocket section 56. In a desired feature, the access opening is located in a portion of the pocket section that is relatively furthest from the waistband edge (e.g. waist edge 20) of the body panel (e.g. body panel 4) on which the pocket section 56 is formed. The free edge region 58 and the access opening can have any operative shape or form. The shape can be linear, curved, angular, rectilinear, curvilinear or the like, as well as combinations thereof. As representatively shown, for example, the contour of the free edge region 58 can have a generally curvilinear shape.

In a particular feature, the free-edge region 58 of the pocket section 56 can have an extending flap section 76 that extends toward the crotch member 50 and away from the waistband edge of the body panel. Additionally, an operative, pocket fastener mechanism 74 can be operatively connected to a bodyside surface of the pocket section 56. In a particular feature, the pocket fastener can be operatively connected to a bodyside surface of the flap section 76. The pocket fastener mechanism 74 can include any operative fastener mechanism, such as a component of an interengaging mechanical fastener, an adhesive fastener, a cohesive fastener, a magnetic fastener, and electromechanical fastener or the like, as well as combinations thereof. The pocket fastener mechanism is operatively configured to provide an attachment that is sufficiently secure to provide for a desire disposal of the article.

In a desired configuration, the pocket fastener mechanism 74 can be a component of an interengaging mechanical fastener. Additionally, the first body panel 4 can have an outer surface, which is provided by a nonwoven fabric and is operatively engageable by the pocket fastener mechanism 74. Accordingly, the pocket fastener mechanism can provide an attachment that is sufficiently secure to hold the article 2 in an operative, closed condition for disposal.

In a further feature, the pocket fastener mechanism 74 can be a hook component of a “hook-and-loop” fastening system. The hook component can have any operative configuration. For example, the hook can have a single prong element, multiple prongs, a mushroom head, a nail head or the like, as well as combinations thereof. Accordingly, a nonwoven fabric or other operative “loop” material on the outer surface of the cooperating body panel (e.g. second body panel 6) or on the outer surface of the crotch member can be operatively engaged by the hook component to provide an attachment that is sufficiently secure to hold the article in an operative closed condition for disposal. Prior to disposal, the pocket fastener mechanism 74 can be configured to operatively engage the outer surface of the crotch member 50 to close the access opening of the pocket section 56 and hold the pocket section in a storage condition.

In another feature, the backsheet layer 68 of the crotch member 50 can have an outward-facing surface which includes a nonwoven fabric. Additionally, the article of the invention can have a configuration in which the pocket fastener mechanism can operatively engage the nonwoven fabric to provide an attachment that is sufficiently secure to hold the article an operative, closed condition for disposal. In a desired arrangement, the pocket fastener mechanism can be a hook component of an interengaging mechanical fastener, and the hook component can operatively engage a nonwoven fabric located on the outward-facing surface of the backsheet layer employed to form the crotch member. The engagement can provide an attachment that is sufficiently secure to hold the article in an operative, closed condition for disposal.

In further aspect of the invention, the pocket section 56 has sufficient size to accommodate a suitable placement of an operative portion of the first body panel 4 therein. In a particular feature, the pocket section can have sufficient size to accommodate a placement of operative portions of the first and second body panels 4 and 6 therein. In another feature, the pocket section 56 can have sufficient size to accommodate a placement of operative portions of the first body panel 4, second body panel 6 and crotch member 50 therein.

To help provide the desired accommodation of the selected portions of the article, the free edge of the pocket section provides an access opening having a lateral opening width 78, and in particular aspects, the lateral opening width can be at least a minimum of about 15 cm. The lateral opening width 78 can alternatively be at least about 16 cm, and can optionally be at least about 17 cm to provide desired benefits. In other aspects, the lateral opening width can be up to a maximum of about 21 cm, or more. The lateral opening width 78 can alternatively be up to about 20 cm, and can optionally be up to about 19 cm to provide desired levels of effectiveness.

The pocket section 56 can also have a pocket depth 80, and in particular aspects, the pocket depth can be at least a minimum of about 10 cm. The pocket depth 80 can alternatively be at least about 11 cm, and can optionally be at least about 12 cm to provide desired benefits. In other aspects, the pocket depth 80 can be up to a maximum of about 15 cm, or more. The pocket depth can alternatively be up to about 14 cm, and can optionally be up to about 13 cm to provide desired levels of effectiveness.

As representatively shown in FIGS. 6 through 6D, the article 2 can be appropriately manipulated, such as by folding and/or rolling, to place the article in a desired disposal configuration.

With reference to FIG. 6, the crotch member 50 that connects between the front and rear body panels 4, 6 can be folded or otherwise turned back on itself along a crotch-fold 86, such that the first side portions 24 of the first body panel 4 are operatively connected or otherwise positioned operatively adjacent to the second side portions 28 of the second body panel 6. This arrangement may already be provided if the article has been previously constructed with side seams. In this position, the body panels can cooperatively provide an article waistband that can substantially encircle a wearer's waist. As representatively shown, the side edge portions of the first and second body panels can be operatively aligned to a position wherein the cooperating side edge portions can be fixedly secured at a seam. Alternatively, the side edge portions of the first and second body panels can be operatively aligned but unaffixed with a seam. With reference to FIG. 6A, a first pair of aligned, adjacently positioned ear portions (30, 32) at one lateral side of the article can be folded or otherwise turned back at a first ear-fold region 92 in a generally lateral movement along a path which rotates the first pair of aligned ear portions in a direction away from the pocket section 56 and pivots the first pair of ear portions about the first ear-fold region. Similarly, a second pair of operatively aligned, adjacently positioned ear portions (30, 32) at the second, laterally-opposed side of the article can also be folded or otherwise turned back along a second ear-fold region 92a in a generally lateral movement along path which rotates and pivots the second pair of aligned ear portions in a direction away from the pocket section 56, as representatively shown in FIG. 6B. Then, as illustrated in FIG. 6C, the group of previously folded or turned-back portions of the body panels can be further folded or otherwise turned back in a generally longitudinal movement which pivots the group of body panel portions about a first end fold region 99 in a path that rotates away from the pocket section. The distal portions 98 of the grouped body panels can then be desirably and operatively arranged to extend beyond the location of the crotch-fold 86. Then, as representatively shown in FIG. 6D, the distal portions 98 of the previously arranged body panels can be tucked or otherwise inserted into the pocket section 56 under the pocket free-edge region 58, and operatively secured with the pocket fastener 74. Accordingly, the article can be discreetly placed in convenient configuration for disposal.

Referring again to FIG. 1, the overall length of the absorbent garment, or article length (AL), can be between about 23 inches and about 40 inches. Of course, the AL can optionally be greater or less than lengths in that range. In various configurations, the length of the absorbent composite (ACL) can be between about 15% and about 95% of the AL, in other aspects, the ACL can be between about 25% and about 80% of the AL, and in further aspects, the ACL can be between about 50% and about 70% of the AL. In various arrangements, each body panel 4, 6 can have a length (BPL1 and BPL2 respectively) that is between about 20% and about 50% of the AL, in particular aspects, BPL1 and BPL2 can each be between about 25% and about 45% of the AL, and in other aspects, BPL1 and BPL2 can each be between about 30% and about 40% of the AL.

In operation, the wearer ordinarily applies the garment 2, which, depending on the configuration thereof, includes pulling the garment up the wearer's legs like underwear or fastening the garment around the wearer like a diaper. As the wearer applies and wears the garment 2, their legs can apply a force to the opposite side edges 52 of the absorbent composite or other crotch member 50 in the crotch region thereof, particularly at the location of the gap 34 between the body panels. The force of the wearer's legs tends to bunch or gather the crotch member 50 between the opposite side edges 52, thereby decreasing the overall width of the absorbent composite and improving the overall fit of the garment on the wearer. In particular, the selected attachment locations 54, 84 at the edge regions of the absorbent composite, do not tend to restrict the gathering of the absorbent composite in the crotch region.

In another aspect, the manufacturer or retailer of such garments provides instructional information to the wearer, for example by way of textual or pictorial indicia on the packaging materials, about how the garment works. For example, the manufacturer or retailer can explain to the end wearer the advantages of the attachment locations, and the attendant ability of the absorbent composite to bunch or gather, as set forth herein, thereby improving the conformance and fit of the garment.

The present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. An absorbent article comprising
a first body panel having a first waistband edge and laterally opposed first side portions;
a separately provided second body panel having a second waistband edge and laterally opposed second side portions; and
a separately provided crotch member which is operatively connected between the first body panel and second body panel; wherein
the first body panel is operatively connected to the crotch member along a contoured attachment zone which thereby provides a pocket section of the first body panel;
the pocket section is operatively unattached to the crotch member and has a free edge region of the first body panel that provides an operative access opening into the pocket section; and
the free-edge region of the pocket section includes an operative, pocket fastener mechanism that is operatively connected to a bodyside surface of the pocket section of the first body panel.

2. An absorbent article as recited in claim 1, wherein the crotch member includes a liquid-permeable topsheet layer, a backsheet layer and an absorbent body sandwiched between the topsheet layer and backsheet layer.

3. An absorbent article as recited in claim 1, wherein the first side portions of the first body panel are operatively connected to the second side portions of the second body panel to provide an article waistband that is configured to substantially encircle a wearers waist.

4. An absorbent article as recited in claim 1, further including at least one side fastener operatively connected to the side portion of at least one body panel, the side fastener configured to secure the absorbent article about the waist of a wearer.

5. An absorbent article as recited in claim 1, wherein
the pocket fastener mechanism is a component of an interengaging mechanical fastener; and
the first body panel has an outer surface, which is provided by a nonwoven fabric and is operatively engageable by the pocket fastener mechanism to provide an attachment that is sufficiently secure to hold the article in an operative, closed condition for disposal.

6. An absorbent article as recited in claim 1, wherein
the pocket fastener mechanism is a hook component of an interengaging mechanical fastener; and
the first body panel has an outer surface provided by a nonwoven fabric which is operatively engageable by the hook component to provide an attachment that is sufficiently secure to hold the article in an operative, closed condition for disposal.

7. An absorbent article as recited in claim 1, wherein the free-edge region of the pocket section has an extending flap section that extends toward the crotch member; and the pocket fastener mechanism that is operatively connected to a bodyside surface of the flap section.

8. An absorbent article as recited in claim 1, wherein the contoured attachment zone has a rectilinear shape.

9. An absorbent article as recited in claim 1, wherein the contoured attachment zone has a curved shape.

10. An absorbent article as recited in claim 1, wherein the contoured attachment zone is generally U-shaped.

11. An absorbent article as recited in claim 1, wherein the backsheet layer of the crotch member has an outward-facing surface which includes a nonwoven fabric.

12. An absorbent article as recited in claim 1, wherein the backsheet layer of the crotch member has an outward-facing surface which includes a nonwoven fabric that is operatively engageable by the pocket fastener mechanism to provide an attachment that is sufficiently secure to hold the article an operative, closed condition for disposal.

13. An absorbent article as recited in claim 1, wherein
the pocket fastener mechanism is a hook component of an interengaging mechanical fastener; and
the backsheet layer of the crotch member has an outward-facing surface which includes a nonwoven fabric that is operatively engageable by the hook component to provide an attachment that is sufficiently secure to hold the article an operative, closed condition for disposal.

14. An absorbent article as recited in claim 1, wherein the pocket fastener mechanism is an adhesive which can provide an attachment that is sufficiently secure to provide for an operative disposal of the article.

15. An absorbent article as recited in claim 1, wherein the pocket section has sufficient size to accommodate a placement of an operative portion of the first body panel therein.

16. An absorbent article as recited in claim 1, wherein the pocket section has sufficient size to accommodate a placement of operative portions of the first and second body panels therein.

17. An absorbent article as recited in claim 1, wherein the free edge of the pocket section provides an access opening having a lateral opening width of at least about 15 cm.

18. An absorbent article as recited in claim 1, wherein the pocket section has a pocket depth of at least about 10 cm.
